(19) United States
(12) Patent Application Publication
(10) Pub. No.: US 2018/0355406 A1
(43) Pub. Date: Dec. 13, 2018

(54) POLYNUCLEIC ACID MOLECULE ENRICHMENT METHODOLOGIES

(71) Applicant: GENETICS RESEARCH, LLC, D/B/A ZS GENETICS, INC., Wakefield, MA (US)

(72) Inventor: William Glover, Wakefield, MA (US)

(21) Appl. No.: 16/007,656

(22) Filed: Jun. 13, 2018

Related U.S. Application Data
(60) Provisional application No. 62/577,851, filed on Oct. 27, 2017.
(60) Provisional application No. 62/526,091, filed on Jun. 28, 2017.
(60) Provisional application No. 62/519,051, filed on Jun. 13, 2017.

(51) Int. Cl. C12Q 1/683; C12Q 1/6886

(52) U.S. Cl. CPC C12Q 1/683; C12Q 2600/156; C12Q 1/6886

(57) ABSTRACT

The invention provides methods of isolating a target nucleic acid in a sample. A polynucleotide region flanking a target nucleic acid may be modified by polymerase extension using modified nucleotides resistant to nuclease degradation to create a modified polynucleotide. Alternatively, an oligonucleotide including the modified nucleotides may be ligated to those regions to create the modified polynucleotide. The sample is exposed to a nuclease, thereby isolating the modified polynucleotide and the target nucleic acid. In other alternatives, terminal phosphates may be removed from a desired portion of a polynucleotide with a double-stranded break to create a modified polynucleotide that is resistant to nuclease degradation, or an epigenetic-binding moiety may be bound to a polynucleotide sequence within or flanking target nucleic acids to sterically inhibit nuclease degradation of the target nucleic acids.

### CROSS-REFERENCE TO RELATED APPLICATIONS

This application claims the benefit of, and priority to, U.S. Provisional Application 62/577,851, filed Oct. 27, 2017, U.S. Provisional Application 62/526,091, filed Jun. 28, 2017, and U.S. Provisional Application 62/519,051, filed Jun. 13, 2017, the contents of each of which are incorporated by reference.

### FIELD OF THE INVENTION

The invention relates to molecular genetics.

### BACKGROUND

Cancer is a leading cause of death, killing millions of people each year. Worldwide, the number of newly diagnosed cancer cases per year is expected to rise to 23.6 million by 2030. Accurate and early diagnosis is essential to improved treatment of cancer. However, early, accurate diagnosis of cancer is difficult when detection and analysis methods, such as sequencing, are time-consuming, expensive, and lack sensitivity.

More sensitive detection methods may allow for earlier detection, or detection that occurs before the disease reaches a stage when treatment is ineffective. Recommending an effective course of treatment is challenging when the diagnostic methods fail to identify the type of cancer. Mutations specific to certain types of cancer can be present in low abundance and difficult to detect without sensitive detection methods. Further, healthcare professionals are unable to accurately monitor the progression of the disease and response to treatment if the detection methods lack sensitivity. Without sensitive detection methods, cancer will continue to kill millions of people annually.

### SUMMARY

The invention provides methods that isolate a target nucleic acid, such as a mutation indicative of cancer, in a sample. Methods of the invention allow for detection of elements present at low quantities, such as mutations specific to certain cancer types, in nucleic acid samples. By isolating the mutations, the invention allows for a greater depth of sequencing coverage when sequencing the isolated regions of interest or target nucleic acids. This allows for increased sampling numbers and reduces the time and costs associated with sequencing.

The sensitivity of the invention makes methods useful for monitoring the progression of disease and determining the stage of cancer. By detecting mutations present at low quantities, cancer or related diseases can be detected at early stages when effective treatment is possible. As such, healthcare professionals may use methods of the invention for an early, accurate diagnosis. Methods of the invention may further be used to predict efficacy of treatment, as progression of the disease may be monitored after treatment. Methods of the invention are also useful for other diagnostic applications that require detection of low-abundance nucleic acids.

Certain embodiments of the invention provide methods for isolating a target nucleic acid. To isolate the target nucleic acid, undesired portions of a polynucleotide that contains the target nucleic acid as well as other unprotected polynucleotides contained in a sample may be selectively degraded/digested by a nuclease, such as an exonuclease. In selective degradation, selectively protected molecules are not degraded, facilitating isolation of the target nucleic acid.

The invention provides various methods to protect the target nucleic acid from nuclease degradation. Through selective protection prior to nuclease-mediated degradation, the target nucleic acid may be isolated from a sample after other unprotected nucleotides are degraded. In an embodiment, selective protection may be achieved by modification of polynucleotide sequences flanking the target nucleic acid using modified nucleotides that are resistant to degradation to create a modified polynucleotide. In one example, the modified nucleotides may be attached to regions flanking the target nucleic acid by a polymerase extension reaction. Alternatively, an oligonucleotide containing the modified nucleotides may be ligated to the regions flanking the target nucleic acid.

In another embodiment, the target nucleic acid may be protected by binding an epigenetic-binding moiety to a polynucleotide sequence within or flanking target nucleic acids in a sample to sterically inhibit nuclease degradation of the target nucleic acids. In an embodiment, methylated nucleotides, may be selectively protected from nuclease degradation. For example, methyl cytosine may be protected from nuclease degradation through steric inhibition by methyl-cytosine binding proteins or methyl-cytosine binding anti-bodies. In some embodiments, unmethylated DNA may be of a pathogen, whereas methylated DNA may be a host or human DNA. In this method, prokaryotic DNA may be enriched from a sample comprising eukaryotic DNA.

In another embodiment, the target nucleic acid may also be protected by dephosphorylating a polynucleotide having at least one double-stranded break flanking a target nucleic acid in a sample to create a modified polynucleotide. For example, terminal phosphates may be removed from regions flanking the target nucleic acid to generate a modified polynucleotide resistant to nuclease degradation. Selective degradation of undesired molecules may be achieved by using nucleases that select for certain epigenomic or non-canonical genomic features associated with undesired molecules, such as methylated DNA.

In another example, target nucleic acids may be isolated by selective degradation of polynucleotides having certain epigenetic modifiers. For example, target nucleic acids may be isolated by preferential degradation of methylated DNA. In this example, a methylcytosine specific endonuclease may digest only DNA that includes methylcytosine bases in a sample, which may leave open, unprotected ends created by the methylcytosine specific endonuclease. When the sample is exposed to an exonuclease, those open, unprotected ends may be degraded, resulting in enrichment of protected, unmethylated and closed-loop molecules.

In one aspect, the invention provides a method for isolating a target nucleic acid. The target nucleic acid may be isolated from a sample by first hybridizing at least one primer to a polynucleotide sequence flanking the target nucleic acid. The primer may be extended using a polymerase and modified nucleotides that are resistant to degradation to create a modified polynucleotide. When the sample now including the modified polynucleotides is exposed to a nuclease, regions of the polynucleotide not protected by the modified nucleotides may be selectively degraded along with other unprotected polynucleotides in the sample. In certain embodiments, the nuclease may be an exonuclease. Through selective degradation, the modified polynucleotide may be isolated.

In an example, an extension reaction may be used to extend a primer hybridized to the polynucleotide sequence flanking the target nucleic acid. The sample may be exposed to a selective nuclease that generates at least one double-stranded break including an overhang prior to hybridizing the primer. The overhang may be a 5′ overhang or a 3′ overhang and an overhang may be generated at one end or both ends of the double-stranded break. For example, an endonuclease may be used to generate the overhang. The selective nuclease may be selected from: a methylation specific nuclease, a methylcytosine-specific endonuclease, a mismatch excision nuclease, a uracil excision nuclease, an abasic site nuclease, a restriction enzyme, and a sequence dependent nuclease.

During primer extension, the polymerase may fill in at least a portion of the overhang with modified nucleotides to create the modified polynucleotide. For example, when a 5′ overhang is generated at a region flanking the target nucleic acid, the 3′ end may be filled in via polymerase extension with the modified nucleotides. Alternatively, an oligonucleotide containing the modified nucleotides may be ligated to the overhang to create the modified polynucleotide, via a ligase.

The modified nucleotides may be any suitable nucleotides that resist nuclease degradation. The modified nucleotides may be used in combination with natural nucleotides. The modified nucleotides may include modified nucleotide triphosphates, alpha-phosphorothioate nucleotide triphosphates, morpholino triphosphates, peptide nucleic acids, peptide nucleic acid analogs, or sugar modified nucleotide triphosphates.

The modified nucleotides may be, for example, 2′-Deoxycytidine-5′-O-(1-Thiotriphosphate), 2′-O-methyl modified nucleotide triphosphate, 2′-fluoro modified nucleotide, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, 2′-O-Methylinosine-5′-Triphosphate, 2′-O-Methyl-2-aminoadenosine-5′-Triphosphate, 2′-O-Methylpseudouridine-5′-Triphosphate, 2′-O-Methyl-5-methyluridine-5′-Triphosphate, 2′-O-Methyl-N6-Methyladenosine-5′-Triphosphate, 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-Fluoro-thymidine-5′-Triphosphate, 2′-Deoxyadenosine-5′-O-(1-Thiotriphosphate), 2′-Deoxycytidine-5′-O-(1-5 Thiotriphosphate), 2′-Deoxyguanosine-5′-O-(1-Thiotriphosphate), 2′-Deoxythymidine-5′-O-(1-Thiotriphosphate), Adenosine-5′-O-(1-Thiotriphosphate), Cytidine-5′-O-(1-Thiotriphosphate), Guanosine-5′-O-(1-Thiotriphosphate), Uridine-5′-O-(1-Thiotriphosphate), 2′,3′-Dideoxyadenosine-5′-O-(1-Thiotriphosphate), 2′,3′-Dideoxycytidine-5′-O-(1-Thiotriphosphate), 2′,3′-Dideoxyguanosine-5′-O-(1-Thiotriphosphate), 3′-Deoxythymidine-5′-10 O-(1-Thiotriphosphate), 3′-Azido-2′,3′-dideoxythymidine-5′-O-(1-Thiotriphosphate), 2′,3′-Dideoxyuridine-5′-O-(1-Thiotriphosphate), 2′-Deoxyadenosine-5′-O-(1-Boranotriphosphate), 2′-Deoxycytidine-5′-O-(1-Boranotriphosphate), 2′-Deoxyguanosine-5′-O-(1-Boranotriphosphate), or 2′-Deoxythymidine-5′-O-(1-Boranotriphosphate).

The modified polynucleotide that is resistant to nuclease degradation may include at least one phosphorothioate linkage, N3′ phosphoramidate linkage, boranophosphate internucleotide linkage, or phosphonoacetate linkage.

The sample may be a blood sample, serum sample, plasma sample, urine sample, saliva sample, semen sample, feces sample, phlegm sample, or liquid biopsy.

In another aspect, the invention provides a method for isolating a target nucleic acid that includes cleaving, in a sequence-specific manner, a polynucleotide sequence flanking a target nucleic acid in a sample to generate at least one double-stranded break flanking the target nucleic acid. Modified nucleotides that are resistant to degradation may be linked to an overhang of the double-stranded break to create a modified polynucleotide. When the sample now including the modified polynucleotides is exposed to a nuclease, regions of the polynucleotide not protected by the modified nucleotides may be selectively degraded along with other unprotected polynucleotides in the sample. In certain embodiments, the nuclease may be an exonuclease. Through selective degradation, the modified polynucleotide may be isolated.

In one embodiment, linking the modified nucleotides includes hybridizing at least one primer to the overhang, and extending the primer using a polymerase and the modified nucleotides to create the modified polynucleotide. In another embodiment, linking the modified nucleotides includes ligating an oligonucleotide comprising the modified nucleotides to the overhang to create the modified polynucleotide.

Cleaving a polynucleotide sequence flanking the target nucleic acid to generate at least one double-stranded break may be performed by a Cas endonuclease complexed with a guide RNA that targets the Cas endonuclease to a region flanking the target nucleic acid. For example, the Cas endonuclease may be Cpf1 and may generate a 5′ overhang at an end of the double-stranded break.

The modified nucleotides may be any suitable nucleotides that resist nuclease degradation. The modified nucleotides may be used in combination with natural nucleotides. The modified nucleotides may include modified nucleotide triphosphates, alpha-phosphorothioate nucleotide triphosphates, morpholino triphosphates, peptide nucleic acids, peptide nucleic acid analogs, or sugar modified nucleotide triphosphates.

The modified polynucleotide that is resistant to nuclease degradation may include at least one phosphorothioate linkage, N3′ phosphoramidate linkage, boranophosphate internucleotide linkage, or phosphonoacetate linkage.

In another aspect, the invention provides a method for isolating a target nucleic acid that includes binding an epigenetic-binding moiety to a polynucleotide sequence within or flanking target nucleic acids in a sample. The epigenetic-binding moiety may sterically inhibit nuclease degradation of the target nucleic acids. When the sample is exposed to a nuclease, regions of the polynucleotide not protected by the epigenetic-binding moiety may be selectively degraded along with other unprotected polynucleotides in the sample. In certain embodiments, the nuclease may be an exonuclease. Through selective degradation, the target nucleic acids may be isolated.

The epigenetic-binding moiety may be any chemical moiety that selectively binds epigenetic modifiers, such as methylated nucleotides. The epigenetic-binding moiety may include, for example, a protein or an antibody. In a preferred embodiment, the epigenetic-binding moiety includes methyl-cytosine binding proteins or methyl-cytosine binding antibodies. The sample may be a blood sample, serum sample, plasma sample, urine sample, saliva sample, semen sample, feces sample, phlegm sample, or liquid biopsy.

In another aspect, the invention provides a method for isolating a target nucleic acid that includes dephosphorylating a polynucleotide having at least one double-stranded break flanking a target nucleic acid in a sample to create a modified polynucleotide. For example, removal of terminal phosphates through dephosphorylation may create a modified polynucleotide resistant to nuclease degradation. When the sample is exposed to a nuclease, regions of the polynucleotide not protected by the epigenetic-binding moiety may be selectively degraded along with other unprotected polynucleotides in the sample. In certain embodiments, the nuclease may be an exonuclease. Through selective degradation, the modified polynucleotide may be isolated. In one embodiment, the method may further include cleaving, in a sequence-specific manner, a polynucleotide sequence flanking the target nucleic acid in the sample to generate the at least one double-stranded break prior to dephosphorylation. In a preferred embodiment, the cleaving may be performed by a Cas endonuclease complexed with a guide RNA that targets the Cas endonuclease to a region flanking the target nucleic acid.

### BRIEF DESCRIPTION OF THE DRAWINGS

FIG. 1 shows primer extension-mediated polynucleic acid enrichment. Extension replication of a polynucleic acid molecule (represented here as dsDNA) region of interest using modified triphosphates, a primer that binds to a sequence flanking the region of interest (a single primer in this instance), and a polymerase generates a modified polynucleic acid molecule that is resistant to nuclease-mediated cleavage. Subsequent exposure of the polynucleic acid mixture to a nuclease, such as an exonuclease, results in digestion of the unprotected polynucleic acid molecules and, thus, enrichment of the region of interest.

FIG. 2 shows protection of Lambda DNA via primer extension. Extension of Lambda DNA template was performed using a polymerase, one primer (Primer 1, generating PEx-1) or two primers (Primers 1 and 8, generating PEx-2), and unmodified nucleotides or modified nucleotides (GaS). Incorporation of modified nucleotides protects the extended Lambda DNA from nuclease-mediated digestion (exo).

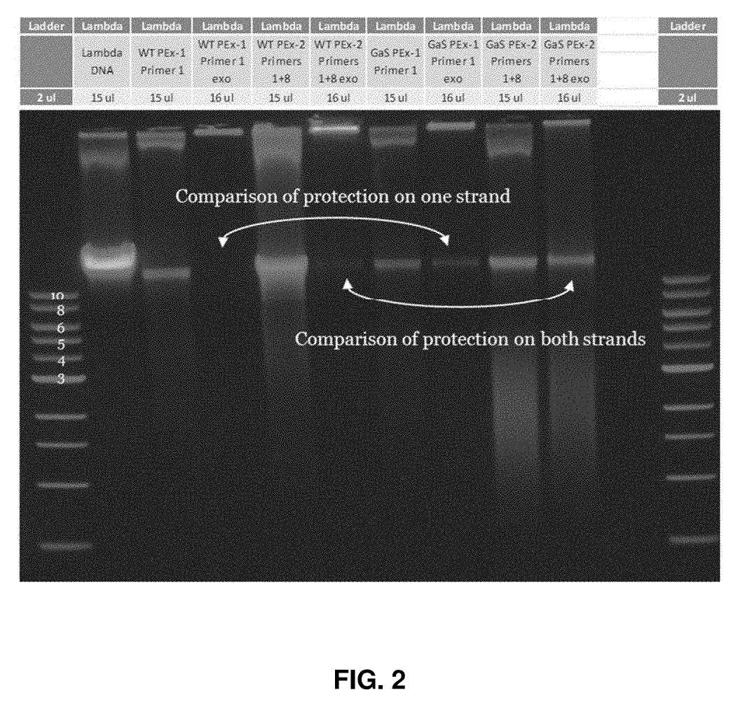

FIG. 3 shows protection of Lambda DNA via primer extension. Extension of Lambda DNA template was performed using a polymerase, one primer (Primer 3, generating PEx-1) or two primers (Primers 3 and 6, generating PEx-2), and unmodified nucleotides or modified nucleotides (GaS). Incorporation of modified nucleotides protects the extended Lambda DNA from nuclease-mediated digestion (exo).

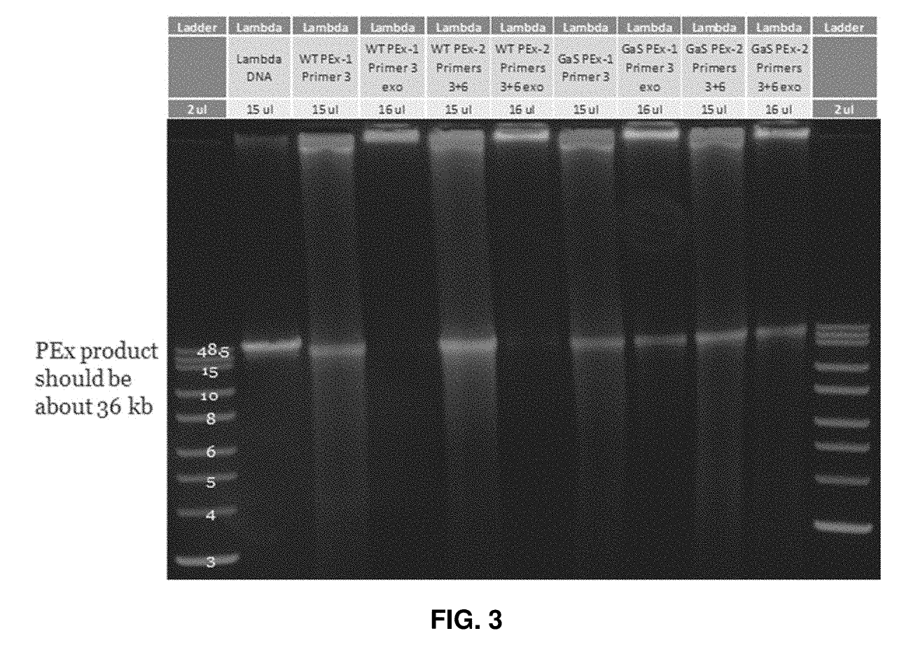

FIG. 4 shows protection of Lambda DNA via primer extension. Extension of Lambda DNA template was performed using a polymerase, one primer (Primer 4, generating PEx-1) or two primers (Primers 4 and 5, generating PEx-2), and unmodified nucleotides or modified nucleotides (GaS). Incorporation of modified nucleotides protects the extended Lambda DNA from nuclease-mediated digestion (exo).

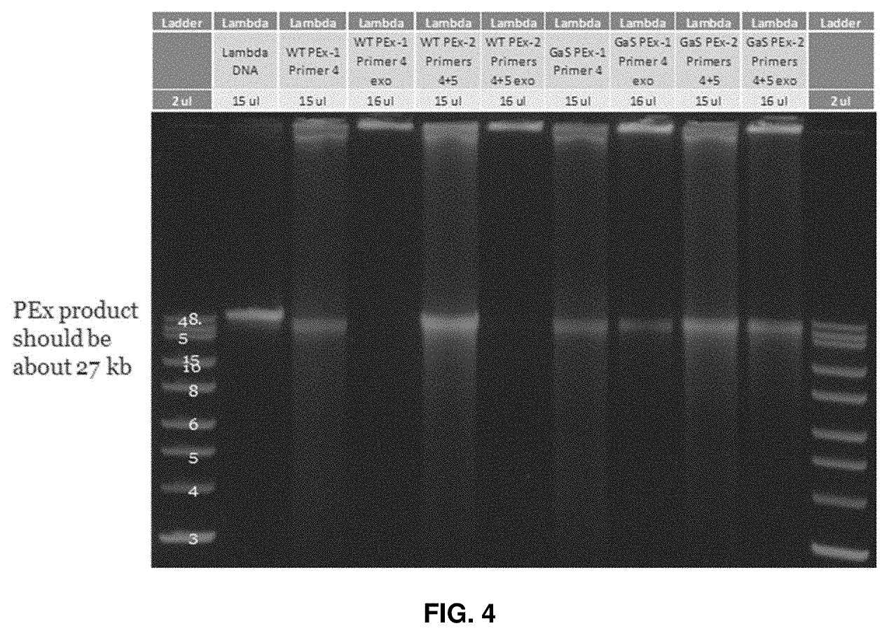

FIG. 5 shows End protection of Lambda DNA via extension. The ends of Lambda DNA have 12-base 5′ overhangs; thus, the 3′ strand can be filled in using a polymerase and nucleotide triphosphates. Incorporating modified nucleotides bases in the 3′ strands of the Lambda DNA protects it from nuclease-mediated digestion.

FIG. 6 shows end protection of Lambda DNA via extension. The ends of Lambda DNA have 12-base 5′ overhangs; thus, the 3′ strand can be filled in using a polymerase and modified nucleotide triphosphates. Incorporating modified nucleotides bases in the 3′ strands of the Lambda DNA protects it from nuclease-mediated digestion.

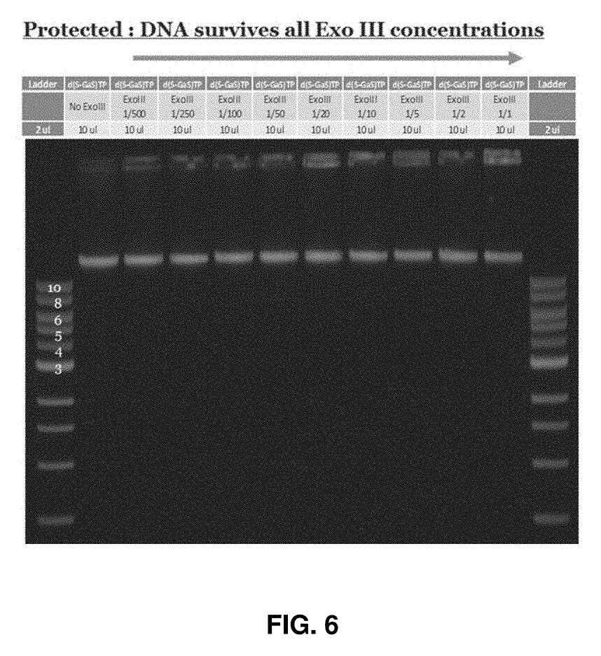

### DETAILED DESCRIPTION

For many polynucleic acid sequencing applications, enrichment is used to reduce or eliminate polynucleic acid molecules that are not of interest and to select for those that are of interest. Applications wherein enrichment is common include the examination of specific copy number variants, single nucleotide polymorphisms, or DNA rearrangements, and the examination of specific “classes” of polynucleic acid molecules (e.g., messenger RNA, noncoding RNA, genomic DNA, exonic genomic DNA, mitochondrial DNA, etc.). By targeting a specific polynucleic acid molecule, one can obtain greater depth of sequencing coverage for regions of interest and increase sampling numbers, thereby reducing the time and costs associated with sequencing.

Previously described enrichment methodologies can be roughly divided into two categories based on how desired polynucleic acid sequences are “captured” or selected from a large polynucleic acid pool: hybridization-based strategies and PCR amplification-based strategies (Kozarewa et al., Curr. Protoc. Mol. Biol. 112, 1-23 (2015); Altmuller et al., Biol. Chem. 395, 231-37 (2014); Mertes et al., Brief Funct. Genomics 10, 374-86 (2011)). Hybridization-based strategies involve the use of DNA or RNA probes or “baits” which are single stranded oligonucleotides that are complementary to the region of interest (or a region flanking the area of interest). These probes hybridize to the region of interest in solution or on a solid support so that one can physically isolate the region of interest and, thereby, enrich the region of interest relative to other regions. PCR-based strategies involve the use of specific primer pairs that are complementary to the region of interest (or a region flanking the area of interest). These primer pairs are used to amplify large amounts of the region of interest and, thereby, enrich the region of interest relative to other regions.

Described herein are novel polynucleic acid molecule enrichment methodologies that are nuclease protection-based strategies, unlike previously described hybridization-based strategies or PCR amplification-based strategies. Nuclease protection-based strategies involve the protection of a polynucleic acid molecule region of interest from nuclease mediated degradation by selective blockage. Application of these nuclease protection-based enrichment methodologies include polynucleic acid sequencing on all long molecule sequencing platforms (e.g., MiSeq (Illumina), NextSeq (Illumina), HiSeq (Illumina), Ion Torrent PGM (Life Technologies), Ion Torrent Proton (Life Technologies), ABI SOLiD (Life Technologies), 454 GS FLX+(Roche), 454 GS Junior (Roche), etc.) as well as short read sequencing platforms.

The term “nucleic acid,” as used herein, refers to a compound comprising a nucleobase and an acidic moiety (e.g., a nucleoside, a nucleotide, or a polymer of nucleotides). As used herein, the terms “polynucleic acid” or “polynucleic acid molecule” are used interchangeably and refer to polymeric nucleic acids (e.g., nucleic acid molecules comprising three or more nucleotides that are linked to each other via a phosphodiester linkage).

Polynucleic acid molecules have various forms. In some embodiments, the polynucleic acid molecule is DNA. In some embodiments, the polynucleic acid molecule is double-stranded DNA. For example, in some embodiments, the DNA is genomic DNA. In other embodiments, the polynucleic acid molecule is single-stranded DNA. In some embodiments, the polynucleic acid molecule is RNA. In some embodiments, the polynucleic acid molecule is double-stranded RNA. In other embodiments, the polynucleic acid molecule is single-stranded RNA.

In some embodiments, the polynucleic acid molecule is contained in or isolated from a biological sample. As used herein, the term “contained in” refers to a polynucleic acid molecule that is within a biological sample. For example, in some embodiments, a polynucleic acid region of interest is protected from nuclease-mediated degradation while the polynucleic acid is within a living biological sample. In other embodiments, a polynucleic acid region of interest is protected from nuclease-mediated degradation whilethe polynucleic acid is within a lysed biological sample.

The term “isolated,” as used herein refers to the separation of a polynucleic acid component of a biological sample from other molecules of a biological sample. For example, in some embodiments, a polynucleic acid region of interest is protected from nuclease-mediated degradation after the polynucleic acid component of a biological sample has been separated from other molecules of a biological sample. Methods of isolating polynucleic acid components from a biological sample are well known to those of skill in the art. Isolation can include partial purification of a polynucleic acid component of a biological sample.

As used herein, the term “biological sample” may refer a cell or a combination of cells. The term “cell” may refer to a prokaryotic cell or a eukaryoticcell. “Prokaryotic cells” include bacteria and archaea. In some embodiments the prokaryotic cell is a bacteria of a phyla selected from Actinobacteria, Aquificae, Armatimonadetes, Bacteroidetes, Caldiserica, Chlamydiae, Chloroflexi, Chrysiogenetes, Cyanobacteria, Deferribacteres, DeinococcusThermus, Dictyoglomi, Elusimicrobia, Fibrobacteres, Firmicutes, Fusobacteria, Gemmatimonadetes, Nitrospirae, Planctomycetes, Proteobacteria, Spirochaetes, Synergistets, Tenericutes, Thermodesulfobacteria, and Thermotogae. In some embodiments the prokaryotic cell is an archaea of a phyla selected from Euryarcheota, Crenarcheota, Nanoarchaeota, Thaumarchaeota, Aigarchaeota, Lokiarchaeota, Thermotogae, and Tenericutes. In some embodiments the eukaryotic cell is a member of a kingdom selected from Protista, Fungi, Plantae, or Animalia.

In some embodiments, the biological sample comprises independent cells (i.e., cells that exist in a single cellular state). In other embodiments, the biological sample comprises cells that exist as part of a multicellular organism. For example, a cell may be located in a transgenic animal or transgenic plant. In some embodiments, the biological sample is a microorganism. In some embodiments, a biological sample is uniform (e.g., made up of the same cell types). In other embodiments, a biological sample is made up of many cell types. In some embodiments, the biological sample comprises blood (or components thereof) or tissue (or components thereof). The term “biological sample” may also refer to a virus. The term “virus” may refer to a DNA virus (e.g., Adenoviridae, Papovaviridae, Parvoviridae, Herpesviridae, Poxiridae, Hepadnaviridae, Anelloviridae, etc.) or an RNA virus (e.g., Reoviridae, Picornaviridae, Calciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornoviridae, Arteriviridae, Hepeviridae, etc.).

The term “virus” may also refer to a phage. As used herein, the term “phage” refers to both bacteriophages and archaeophages. “Bacteriophage” refers to a virus that infects bacteria. “Archaeophage” refers to a virus that infects archaea. Bacteriophages and archaeophages are obligate intracellular parasites that multiply inside a host cell by making use of some or all of the cell's biosynthetic machinery. In some embodiments a phage is a member of an order selected from Caudovirales, Microviridae, Corticoviridae, Tectiviridae, Leviviridae, Cystoviridae, Inoviridae, Lipothrixviridae, Rudiviridae, Plasmaviridae, and Fuselloviridae. In some embodiments the phage is a member of the order Caudovirales and is a member of a family selected from Myoviridae, Siphoviridae, and Podoviridae.

The biological sample can contain or be suspected of containing one or more pathogens or polynucleic acid molecules of one or more pathogens.

As used herein, the term “region of interest” refers to the region of a polynucleic acid that one seeks to enrich relative to other polynucleic acid regions. The length of regions of interest can be of various lengths. For example, in some embodiments, the polynucleic acid molecule region of interest is at least 10,000 nucleotides or base pairs in length, such as 20,000, 25,000, 30,000, 40,000, 50,000, 60,000, 70,000, 80,000, 90,000, 100,000, or more nucleotides or base pairs in length. In some additional embodiments, the polynucleic acid molecule region of interest is 10,000 to 50,000, 50,000 to 100,000, or 100,000 to 1,000,000 nucleotides or base pairs in length, or even longer. In other embodiments, the polynucleic acid molecule region of interest is as few as five nucleotides or base pairs in length, or approximately 180 base pairs in length.

The term “nuclease,” as used herein, refers to an agent, for example, a protein, capable of cleaving a phosphodiester bond connecting two nucleotide residues in a polynucleic acid molecule. The term “nuclease” includes endonucleases, exonucleases, and agents that exhibit both endonuclease and exonuclease activity. As used herein, the term endonuclease refers to a nuclease that is capable of cleaving a phosphodiester bond within a polynucleic acid molecule. Specific endonucleases include, but are not limited to, restriction endonucleases (e.g., EcoRI, BamHI, HindIII, etc.), DNase I, DNase II, Micrococcal nuclease, Mung Bean nuclease, RNase A, RNase H, RNase III, RNase L, RNase P, RNase PhyM, RNase T1, RNase T2, RNase U2, RNase V, and RNA-guided endonucleases (e.g., CRISPR/Cas proteins). Nuclease also includes methyl-cystosine sensitive nucleases such as McrBC. As used herein, the term exonuclease refers to a nuclease that is capable of cleaving a phosphodiester bond at the end of a polynucleic acid molecule. Specific exonucleases include, but are not limited to, T7 exonuclease, T5, exonuclease, lambda exonuclease, Exonuclease I, Exonuclease III, Exonuclease V, Exonuclease VII, ExonucleaseVIII, Exonuclease T, RNase PH, RNase R, RNase T, Oligoribonuclease, Exoribonuclease I, Exoribonuclease II, and PNPase. In some embodiments, the polynucleic acid molecule and the modified polynucleic acid molecule are contacted with at least one endonuclease. In other embodiments, the polynucleic acid molecule and the modified polynucleic acid molecule are contacted with at least one exonuclease. In other embodiments, the polynucleic acid molecule and the modified polynucleic acid molecule are contacted with at least one agent that exhibits endonuclease and exonuclease activity. In other embodiments, the polynucleic acid molecule and the modified polynucleic acid molecule is contacted with a combination of at least one endonuclease, at least one exonuclease, and/or at least one agent that exhibits endonuclease and exonuclease activity.

As used herein, the terms “protection” or “protecting” with respect to a region of interest refer to a decrease in the region of interest's susceptibility to nuclease-mediated cleavage by at least 20%, 25%, 30%, 40%, 50%, 60%, 70%, 80%, 90% or up to 100% relative to other polynucleic acid regions. Methods of measuring and comparing levels of nuclease-mediated cleavage are known to those skilled in the art. In some embodiments, the region of interest is protected from all nucleases. In some embodiments, the region of interest is protected from all exonucleases. In other embodiments, the region of interest is protected from all endonucleases. In still other embodiments, the region of interest is protected from a subset of exonucleases or endonucleases. In other embodiments, the region of interest is protected from a single exonuclease or endonuclease.

The term, “modified nucleotide triphosphate” as used herein refers to any nucleotide triphosphate compound whose composition differs from natural occurring nucleotide triphosphates and whose incorporation into a polynucleic acid molecule renders the polynucleic acid molecule more resistant to nuclease-mediated cleavage relative to a polynucleic acid molecule that does not have incorporated modified bases. Naturally-occurring nucleoside triphosphates include adenosine triphosphate, guanosine triphosphate, cytidine triphosphate, 5-methyluridine triphosphate, and uridine triphosphate. Examples of modified nucleotides triphosphates that meet these requirements are known to those of skill in the art (Deleavey and Damha Chem. Biol. 19, 937-54 (2012); Monia et al. J. Biol. Chem. 271, 14533-40 (1996)).

In some embodiments, at least one of the one or more types of modified nucleotide triphosphates is an alpha-phosphorothioate nucleotide triphosphate. In some embodiments, the alpha-phosphorothioate nucleotide triphosphate is selected from 2′-Deoxyadenosine-5′-O-(1-Thiotriphosphate), 2′-Deoxycytidine-5′-O-(1-Thiotriphosphate), 2′-Deoxyguanosine-5′-O-(1-Thiotriphosphate), 2′-Deoxythymidine-5′-O-(1-Thiotriphosphate), Adenosine-5′-O-(1-Thiotriphosphate), Cytidine-5′-O-(1-Thiotriphosphate), Guanosine-5′-O-(1-Thiotriphosphate), Uridine-5′-O-(1-Thiotriphosphate), 2′,3′-Dideoxyadenosine-5′-O-(1-Thiotriphosphate), 2′,3′-Dideoxycytidine-5′-O-(1-Thiotriphosphate), 2′,3′-Dideoxyguanosine-5′-O-(1-Thiotriphosphate), 3′-Deoxythymidine-5′-O-(1-Thiotriphosphate), 3′-Azido-2′,3′-dideoxythymidine-5′-O-(1-Thiotriphosphate), 2′,3′-Dideoxyuridine-5′-O-(1-Thiotriphosphate), 2′-Deoxyadenosine-5′-O-(1-Boranotriphosphate), 2′-Deoxycytidine-5′-O-(1-Boranotriphosphate), 2′-Deoxyguanosine-5′-O-(1-Boranotriphosphate), or 2′-Deoxythymidine-5′-O-(1-Boranotriphosphate). In some embodiments, the alpha-phosphorothioate is 2′-Deoxycytidine-5′-O-(1-Thiotriphosphate).

In some embodiments, at least one of the one or more types of modified nucleotide triphosphates is a morpholino triphosphate. In some embodiments, at least one of the one or more types of modified nucleotide triphosphates is a peptide nucleic acid or a peptidenucleic acid analog.

In some embodiments, at least one of the one or more types of modified nucleotide triphosphates is a sugar modified nucleotide triphosphate. In some embodiments, the sugar modified nucleotide triphosphate is a 2′ O-methyl modified nucleotide triphosphate. In some embodiments, the 2′ O-methyl modified nucleotide triphosphate is selected from 2′-O-Methyladenosine-5′-Triphosphate, 2′-O-Methylcytidine-5′-Triphosphate, 2′-OMethylguanosine-5′-Triphosphate, 2′-O-Methyluridine-5′-Triphosphate, 2′-O-Methylinosine-5′-Triphosphate, 2′-O-Methyl-2-aminoadenosine-5′-Triphosphate, 2′-O-Methylpseudouridine-5′-Triphosphate, 2′-O-Methyl-5-methyluridine-5′-Triphosphate, or 2′-O-Methyl-N6-Methyladenosine-5′-Triphosphate.

In some embodiments, the sugar modified nucleotide triphosphate is a 2′ fluoro modified nucleotide triphosphate. In some embodiments, the 2′ fluoro modified nucleotide triphosphate is selected from 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, or 2′-Fluoro-thymidine-5′-Triphosphate. In some embodiments, the modified nucleotide triphosphate is biotinylated. In some embodiments, the biotin can be conjugated with moiety that blocks nuclease-mediated digestion.

The term “polymerase” as used herein, refers to an agent, for example, a protein, that is capable of performing primer-dependent polynucleic acid synthesis. Examples of polymerases are well known to those of skill in the art. In some embodiments, the polymerase can utilize single-stranded DNA, double-stranded DNA, single-stranded RNA, double-stranded RNA, and/or a DNA/RNA hybrid as a substrate. As used herein, the term DNA/RNA hybrid refers to a polynucleic acid molecule comprising a DNA molecule hybridized to an RNA molecule. In some embodiments, the polymerase can utilize multiple substrates. For example, in some embodiments, the polymerase can utilize single-stranded DNAs and single-stranded RNAs as a template. In some embodiments, the polymerase does not require double-stranded DNA as substrate. In some embodiments, the polymerase is an RNA polymerase. In other embodiments, the polymerase is a DNA polymerase. In some embodiments, the polymerase is a reverse transcriptase, in which case the product is a cDNA comprising modified nucleotide triphosphates.

The term “phosphatase” as used herein, refers to an agent, for example, a protein, that is capable of removing the terminal phosphate from a polynucleic acid molecule. Examples of polymerases are well known to those of skill in the art, such as calf intestinal alkaline phosphatase (CIP), or shrimp alkaline phosphatase (rSAP). In some embodiments, the phosphatase can utilize single-stranded DNA, double-stranded DNA, single-stranded RNA, double-stranded RNA, and/or a DNA/RNA hybrid as a substrate. In some embodiments, the phosphatase can utilize multiple substrates. For example, in some embodiments, the phosphatase can utilize single-stranded DNAs and single-stranded RNAs as a template. In some embodiments, the phosphatase does not require double-stranded DNA as substrate.

The term “modified polynucleic acid molecule” as used herein refers to a polynucleic acid molecule comprising modified nucleotides. The abundance of modified nucleotides may vary between modified polynucleic acid molecules. For example, in some embodiments, less than 25% of the nucleotides in a modified polynucleic acid molecule are modified nucleotides. In other embodiments, at least 25%, at least 30%, at least 40%, at least 50%, at least 60%, at least 70%, at least 80%, at least 90%, or 100% of the nucleotides in a modified polynucleic acid molecule are modified nucleotides. In some embodiments, the modified polynucleic acid molecule comprises at least one phosphorothioate linkage, N3′ phosphoramidate linkage, boranophosphate internucleotide linkage, or phosphonoacetate linkage.

The term “modified polynucleic acid molecule” as used herein also refers to a dephosphorylated polynucleic acid molecule. In some embodiments, the modified polynucleic acid molecule comprises a single stranded dephosphorylated polynucleic acid molecule. In other embodiments, the modified polynucleic acid molecule comprises a double stranded dephosphorylated polynucleic acid molecule in which one or both strands are dephosphorylated. In some embodiments, the modified polynucleic acid molecule is single-stranded DNA (including cDNA), double-stranded DNA (including cDNA), single-stranded RNA, double-stranded RNA, or a complex of DNA and/or RNA. For example, in some embodiments, one strand of a double-stranded DNA molecule will comprise modified nucleotides, while the other strand does not. In other embodiments, both strands of a double-stranded DNA molecule will comprise modified nucleotides. In other embodiments, one strand of a double-stranded RNA molecule will comprise modified nucleotides, while the other strand does not. In other embodiments, both strands of a double-stranded RNA molecule will comprise modified nucleotides. In other embodiments, the modified polynucleic acid molecule comprises a DNA/RNA hybrid in which either the DNA or the RNA comprises modified nucleotides. In other embodiments, the modified polynucleic acid molecule comprises a DNA/RNA hybrid in which both the DNA and the RNA comprise modified nucleotides. In some embodiments, the modified polynucleic acid molecule is a combination of one or more single-stranded DNAs, double-stranded DNAs, single-stranded RNAs, double-stranded RNAs, or DNA/RNA hybrids.

As used herein, the term “resistant to nuclease-mediated cleavage” refers to a decrease in the modified polynucleic acid's susceptibility to nuclease-mediated cleavage by at least 20%, 25%, 30%, 40%, 50%, 60%, 70%, 80%, 90% or up to 100% relative to a non-modified polynucleic acid molecule. Methods of measuring and comparing levels of nuclease-mediated cleavage are known to those skilled in the art. In some embodiments, the modified polynucleic acid molecule is resistant to all nucleases. In some embodiments, the modified polynucleic acid molecule is resistant to all exonucleases. In other embodiments, the polynucleic acid molecule is resistant to all endonucleases. In still other embodiments, the modified polynucleic acid molecule is resistant to a subset of exonucleases or endonucleases. In other embodiments, the modified polynucleic acid molecule is resistant to a single exonuclease or endonuclease.

While the concentrations of the components utilized in the embodiments disclosed herein (e.g., the modified nucleotide triphosphates, the primer(s), and the polynucleic acid molecules) may vary, the methods can utilize any effective amount of the components. As such, the contents of the reaction mixtures and the reaction incubation times may vary. “Any effective amount of the components” refers to any amount that, when combined, results in the enrichment of at least 50%, 100%, 500%, 1000%, 10,000%, 100,000%, 1,000,000% or more than 1,000,000% in the level of a polynucleic acid region of interest relative to other polynucleic acid molecules.

Described herein are polynucleic acid molecule enrichment methodologies whereby an undesired selection of polynucleic acid molecule molecules is selectively degraded by nuclease-mediated degradation and a desired selection of polynucleic acid molecule is selectively protected from nuclease-mediated degradation. Selective degradation of undesired molecules is effected by using nucleases that select for certain epigenomic or non-canonical genomic features associated with undesired molecules. Selective protection is effected by modification of the desired portion using modified nucleotide triphosphates, removal of terminal phosphates from a desired portion of a polynucleic acid molecule, ligation of an oligonucleotide having modified nucleotide triphosphates to a desired portion of a polynucleic acid molecule, or steric blocking of nuclease function by sequence-specific or feature-specific binding of a blocking moeity. Application of these enrichment methodologies include polynucleic acid sequencing on all sequencing platforms.

### Extension-Mediated Polynucleic Acid Molecule Enrichment

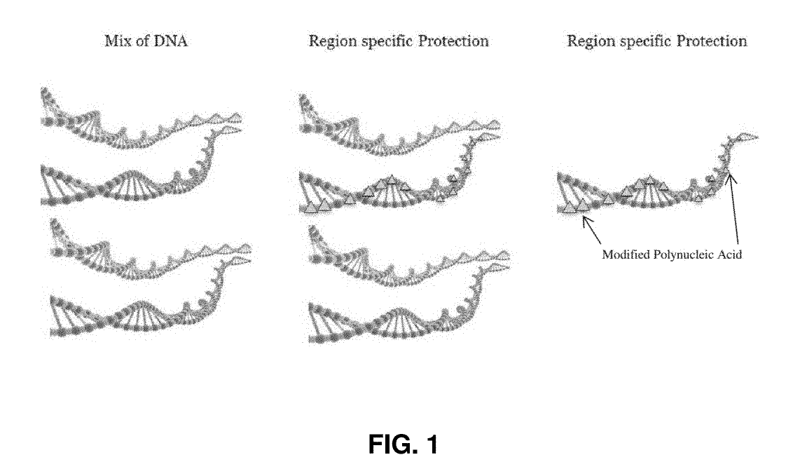

In one aspect, a polynucleic acid region of interest is selectively blocked from nuclease digestion by extension of the region of interest using modified nucleotide triphosphates. Extension of a polynucleic acid molecule region of interest using modified triphosphates generates a modified polynucleic acid molecule that is resistant to nuclease-mediated cleavage. Subsequent exposure of the polynucleic acid mixture to a nuclease results in digestion of the unprotected polynucleic acid molecules and, thus, enrichment of the region of interest (FIG. 1). In some embodiments, a nucleic acid mixture is exposed to a nuclease that selectively degrades nucleic acid polymers with certain epigenetic characteristics. For example, an endonuclease that acts on DNA comprising methyl-cytosine bases but is inactive on DNA without methyl-cytosine bases.

In other embodiments, enrichment of a polynucleic acid molecule region of interest that has at least one 5′ overhang comprises protecting the region of interest by contacting the polynucleic acid molecule with at least one polymerase, extending the 3′ end to fill in at least a portion of the overhang using a polymerase and one or more types of modified nucleotide triphosphates, wherein the extension of the 3′ end to fill in at least a portion of the overhang with the one or more types of modified nucleotide triphosphates generates a modified polynucleic acid molecule that lacks a 5′ overhang or has a smaller 5′ overhang and that is resistant to nuclease-mediated cleavage, and contacting the polynucleic acid molecule and the modified polynucleic acid molecule with a nuclease, thereby digesting the polynucleic acid molecule outside of the region of interest.

In other embodiments, enrichment of a polynucleic acid molecule region of interest that has either no overhang or at least one 3′ overhang comprises protecting the region of interest by contacting the polynucleic acid molecule with at least one polymerase, extending the 3′ end to create a 3′ “tail” using a polymerase and one or more types of modified nucleotide triphosphates, wherein the extension of the 3′ end with the one or more types of modified nucleotide triphosphates generates a modified polynucleic acid molecule that is resistant to nuclease-mediated cleavage, and contacting the polynucleic acid molecule and the modified polynucleic acid molecule with a nuclease, thereby digesting the polynucleic acid molecule outside of the region of interest.

As used herein, the term “overhang” refers to a stretch of unpaired nucleotides at the end of a double stranded polynucleic acid molecule. The length of an overhang can vary. In some embodiments, the overhang is a short as a single nucleotide. In other embodiments, the overhang is between about 1 and 15 nucleotides in length. In other embodiments, the overhang is between about 15 and 100 nucleotides in length. In other embodiments, the overhang is greater than 100 nucleotides in length.

### Polynucleotide Enrichment Mediated by 3′ Extension of 5′ Overhang

According to one aspect, methods for enrichment of a polynucleic acid molecule region of interest are provided. The methods include contacting the double-stranded polynucleic acid molecule which comprises at least one 5′ overhang flanking the region of interest, extending the at least one 5′ overhang with a polymerase and one or more types of modified nucleotide triphosphates, wherein the extension of the at least one 5′ overhang with the one or more types of modified nucleotide triphosphates generates a modified polynucleic acid molecule that is resistant to nuclease-mediated cleavage, and contacting the polynucleic acid molecule and the modified polynucleic acid molecule with a nuclease to digest the polynucleic acid molecule 5′ and 3′ to the modified polynucleic acid, thereby digesting the polynucleic acid molecule outside of the region of interest. In some embodiments, two 5′ overhangs on different strands of the polynucleic acid molecule are thusly modified.

### Polynucleotide Enrichment Mediated by Preferential Digestion of Methylated DNA

According to another aspect, methods for enrichment of a polynucleic acid molecule are provided. The methods include contacting the polynucleic acid molecule with at least one endonuclease which selectively acts on molecules with certain epigenomic properties. For example, using a methylcytosine-specific endonuclease will digest only DNA comprising methylcytosine nucleobases. Subsequently treating the sample with exonuclease(s) will degrade the molecules in which open, unprotected ends were created by the methylcytosine specific endonuclease. Protected molecules, molecules without methylcytosine bases, and molecules comprising closed-loop molecules will not be digested by the exonuclease(s), resulting in enrichment of protected, unmethylated and closed-loop molecules. In some embodiments the unmethylated DNA is a pathogen. In some embodiments, the methylated DNA is host or human DNA. In this method, prokaryotic DNA can be enriched from a sample comprising eukaryotic DNA.

### Polynucleotide Enrichment Mediated by Preferential Digestion of Methylated DNA

In some embodiments, the sample is digested with a methyl-cytosine specific endonuclease after protection is provided. This will create unprotected ends on DNA molecules that comprise methyl-cytosine bases only. Methyl-cytosine specific nucleases can be individual reagents, or combinations of reagents. Nucleases can be organic, inorganic, or combinations. Subsequent exonuclease digestion will preferentially degrade methylated DNA, leaving unmethylated DNA undigested. In some embodiments, the unmethylated DNA is a pathogen. In some embodiments, the methylated DNA is host or human DNA. In this method, prokaryotic DNA can be enriched from a sample comprising eukaryotic DNA.

### Polynucleotide Enrichment Mediated by Non-Templated 3′ Extension

According to another aspect, methods for enrichment of a polynucleic acid molecule region of interest provided. The methods include contacting the polynucleic acid molecule with at least one non-templating polymerase, such as terminal deoxynucleotidyltransferase, and extending the region of interest using the polymerase and one or more types of modified nucleotide triphosphates, wherein the extension of the region of interest with the one or more types of modified nucleotide triphosphates generates a modified polynucleic acid molecule that has a 3′ “tail” and that is resistant to nuclease-mediated cleavage, and contacting the polynucleic acid molecule and the modified polynucleic acid molecule with a nuclease, thereby digesting the polynucleic acid molecules outside of the region of interest. The 3′ end may originally be recessed, blunt or 3′ overhanging.

### Polynucleotide Enrichment Mediated by Preferential Protection of Methylated DNA

In other embodiments, nucleic acid polymers are sterically protected from nuclease degradation by conjugation with methyl-cytosine binding proteins or methyl-cytosine binding anti-bodies. This steric protection from nuclease can be in addition to chemical modification or instead of chemical modification. In some embodiments, steric protection can be provided by epigenetic binding moieties other than those that bind to methyl-cytosine, including the well-known nucleotide modifications observed in nature.

According to another aspect, methods for enrichment of a double-stranded polynucleic acid molecule region of interest are provided. The methods include contacting the polynucleic acid molecule with at least one CRISPR/Cas complex that binds to a sequence of the double-stranded polynucleic acid molecule flanking the region of interest, wherein the contacting of the polynucleic acid molecule with the at least one CRISPR/Cas complex generates at least one double-strand break flanking the region of interest, dephosphorylating the polynucleic acid molecule with at least one double-strand break using a phosphatase, wherein the dephosphorylation of the polynucleic acid molecule with at least one double-strand break generates a modified polynucleic acid molecule that is resistant to nuclease-mediated cleavage, and contacting the polynucleic acid molecule and the modified polynucleic acid molecule with a nuclease, thereby digesting the polynucleic acid molecule outside of the region of interest.

In some embodiments, the methods also include selecting the sequence of the doublestranded polynucleic acid molecule bound by the CRISPR/Cas complex so that the overhang has at least one type of nucleotide that is not present its complementary overhang sequence.

In some embodiments, the double-strand break comprises a 5′ overhang or a 3′ overhang at the ends of the polynucleic acid molecule.

In some embodiments, the polynucleic acid molecule comprises two double-strand breaks flanking the region of interest.

In some embodiments, the CRISPR/Cas complex comprises Cpf1. In other embodiments two nicking endonucleases are used to create two staggered nicks in close proximity on opposite strands of the polynucleic acid.

In some embodiments, the modified polynucleic acid molecule includes at least one phosphorothioate linkage, N3′ phosphoramidate linkage, boranophosphate internucleotide linkage, or phosphonoacetate linkage.

In some embodiments, at least one of the one or more types of modified nucleotide triphosphates is an alpha-phosphorothioate nucleotide triphosphate.

In some embodiments, the alpha-phosphorothioate nucleotide triphosphate is selected from 2′-Deoxyadenosine-5′-O-(1-Thiotriphosphate), 2′-Deoxycytidine-5′-O-(1-Thiotriphosphate), 2′-Deoxyguanosine-5′-O-(1-Thiotriphosphate), 2′-Deoxythymidine-5′-O-(1-Thiotriphosphate), Adenosine-5′-O-(1-Thiotriphosphate), Cytidine-5′-O-(1-Thiotriphosphate), Guanosine-5′-O-(1-Thiotriphosphate), Uridine-5′-O-(1-Thiotriphosphate), 2′,3′-Dideoxyadenosine-5′-O-(1-Thiotriphosphate), 2′,3′-Dideoxycytidine-5′-O-(1-Thiotriphosphate), 2′,3′-Dideoxyguanosine-5′-O-(1-Thiotriphosphate), 3′-Deoxythymidine-5′-O-(1-Thiotriphosphate), 3′-Azido-2′,3′-dideoxythymidine-5′-O-(1-Thiotriphosphate), 2′,3′-Dideoxyuridine-5′-O-(1-Thiotriphosphate), 2′-Deoxyadenosine-5′-O-(1-Boranotriphosphate), 2′-Deoxycytidine-5′-O-(1-Boranotriphosphate), 2′-Deoxyguanosine-5′-O-(1-Boranotriphosphate), or 2′-Deoxythymidine-5′-O-(1-Boranotriphosphate). In some embodiments, the alpha-phosphorothioate nucleotide triphosphate is 2′-Deoxycytidine-5′-O-10 (1-Thiotriphosphate).

In some embodiments, at least one of the one or more types of modified nucleotide triphosphates is a morpholino triphosphate.

In some embodiments, at least one of the one or more types of modified nucleotide triphosphates is a peptide nucleic acid or a peptide nucleic acid analog.

In some embodiments, at least one of the one or more types of modified nucleotide triphosphates is a sugar modified nucleotide triphosphate. In some embodiments, the sugar modified nucleotide triphosphate is a 2′ O-methyl modified nucleotide triphosphate. In some embodiments, the 2′ O-methyl modified nucleotide triphosphate is selected from 2′-OMethyladenosine-5′-Triphosphate, 2′-O-Methylcytidine-5′-Triphosphate, 2′-O-20 Methylguanosine-5′-Triphosphate, 2′-O-Methyluridine-5′-Triphosphate, 2′-O-Methylinosine-5′-Triphosphate, 2′-O-Methyl-2-aminoadenosine-5′-Triphosphate, 2′-O-Methylpseudouridine-5′-Triphosphate, 2′-O-Methyl-5-methyluridine-5′-Triphosphate, or 2′-O-Methyl-N6-Methyladenosine-5′-Triphosphate. In some embodiments, the sugar modified nucleotide triphosphate is a 2′ fluoro modified nucleotide triphosphate.

In some embodiments, the 2′ fluoro modified nucleotide triphosphate is selected from 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, or 2′-Fluoro-thymidine-5′-Triphosphate.

In some embodiments of any of the foregoing methods, the polynucleic acid molecule region of interest is between 10,000 to 50,000, 50,000 to 100,000, 100,000 to 1,000,000, or longer, nucleotides or base pairs in length.

In some embodiments of any of the foregoing methods, the polynucleic acid molecule is contained in or isolated from a biological sample. In some embodiments, the biological sample comprises blood or tissue. In some embodiments, the biological sample comprises microorganisms. In some embodiments, the biological sample is purified.

In some embodiments of any of the foregoing methods, the polynucleic acid molecule is DNA. In some embodiments, the DNA is genomic DNA.

### Polynucleic Acid Molecule Enrichment Following CRISPR/Cas Digestion

In one aspect, a polynucleic acid region of interest is selectively blocked from nuclease digestion following CRISPR/Cas digestion. In some embodiments, enrichment of a double stranded polynucleic acid molecule region of interest comprises contacting the polynucleic acid molecule with at least one CRISPR/Cas complex that binds to a sequence of the double stranded polynucleic acid molecule flanking the region of interest, wherein the contacting of the polynucleic acid molecule with the at least one CRISPR/Cas complex generates at least one double strand break flanking the region of interest, contacting the polynucleic acid molecule with at least one double strand break with a ligase and a double stranded oligonucleotide comprising modified nucleotides, wherein the contacting of the polynucleic acid molecule with at least one double strand break with a ligase and a double stranded oligonucleotide covalently links the region of interest with the double stranded oligonucleotide and generates a modified polynucleic acid molecule that is resistant to nuclease-mediated cleavage, and contacting the polynucleic acid molecule and the modified polynucleic acid molecule with a nuclease, thereby digesting the polynucleic acid molecule outside of the region of interest. In some embodiments, a single-stranded oligonucleotide can be ligated in place of the double-stranded oligonucleotide to generate a modified polynucleic acid molecule that is resistant to nuclease-mediated cleavage, and optionally the overhang created by the single-stranded oligonucleotide can be filled in using a polymerase as described elsewhere herein.

In other embodiments, enrichment of a double-stranded polynucleic acid molecule region of interest includes contacting the polynucleic acid molecule with at least one CRISPR/Cas complex that binds to a sequence of the double-stranded polynucleic acid molecule flanking the region of interest. This contacting of the polynucleic acid molecule with the at least one CRISPR/Cas complex generates at least one double-strand break flanking the region of interest, and the double-strand break comprises overhangs at the ends of the polynucleic acid molecule. The polynucleic acid molecule with at least one double-strand break then is contacted with a polymerase and one or more types of nucleotide triphosphates, wherein at least one type of nucleotide triphosphate confers resistance to nuclease cleavage and is complementary to a nucleotide in the overhang, such that the polymerase fills in the overhangs with the nucleotide triphosphates, including at least one nucleotide triphosphate that confers resistance to nuclease cleavage, and thereby generates a modified polynucleic acid molecule that is resistant to nuclease-mediated cleavage. The polynucleic acid molecule and the modified polynucleic acid molecule comprising the region of interest then are contacted with an exonuclease, thereby digesting the unprotected polynucleic acid molecule, while the modified, protected polynucleic acid molecule comprising the region of interest is not digested.

In some embodiments, the enrichment can further include selecting the sequence of the double-stranded polynucleic acid molecule bound by the CRISPR/Cas complex so that the overhang has at least one type of nucleotide that is not present its complementary overhang sequence. In some embodiments, the overhang is selected such that none of the nucleotides present in the overhang are the same as the nucleotides present in its complementary overhang sequence.

In some embodiments, the double-strand break can include a 5′ overhang or a 3′ overhang at the ends of the polynucleic acid molecule. In some embodiments, the polynucleic acid molecule comprises two double-strand breaks flanking the region of interest. In other embodiments, enrichment of a double stranded polynucleic acid molecule region of interest comprises contacting the polynucleic acid molecule with at least one CRISPR/Cas complex that binds to a sequence of the double stranded polynucleic acid molecule flanking the region of interest, wherein the contacting of the polynucleic acid molecule with the at least one CRISPR/Cas complex generates at least one double strand break flanking the region of interest, dephosphorylating the polynucleic acid molecule with at least one double strand break using a phosphatase, wherein the dephosphorylation of the polynucleic acid molecule with at least one double strand break generates a modified polynucleic acid molecule that is resistant to nuclease-mediated cleavage, and contacting the polynucleic acid molecule and the modified polynucleic acid molecule with a nuclease, thereby digesting the polynucleic acid molecule outside of the region of interest.

As used herein, the term “CRISPR/Cas complex” refers to a CRISPR/Cas protein that is bound to a small guide RNA. As used herein, the term “CRISPR/Cas protein” refers to an RNA-guided DNA endonuclease, including, but not limited to, Cas9, Cpf1, C2c1, and C2c3 and each of their orthologs and functional variants. CRISPR/Cas protein orthologs have been identified in many species and are known or recognizable to those of ordinary skill in the art. For example, Cas9 orthologs have been described in various species, including, but not limited to Bacteroides coprophilus (e.g., NCBI Reference Sequence: WP_008144470.1), Campylobacter jejuni susp. jejuni (e.g., GeneBank: AJP35933.1), Campylobacter lari (e.g., GeneBank: AJD02827.1), Fancisella novicida (e.g., UniProtKB/Swiss-Prot: A0Q5Y3.1), Filifactor alocis (e.g., NCBI Reference Sequence: WP_083799662.1), Flavobacterium columnare (e.g., GeneBank: AMA50561.1), Fluviicola taffensis (e.g., NCBI Reference Sequence: WP_013687888.1), Gluconacetobacter diazotrophicus (e.g., NCBI Reference Sequence: WP_041249387.1), Lactobacillus farciminis (e.g., NCBI Reference Sequence: WP_010018949.1), Lactobacillus johnsonii (e.g., GeneBank: KXN76786.1), Legionella pneumophila (e.g., NCBI Reference Sequence: WP_062726656.1), Mycoplasma gallisepticum (e.g., NCBI Reference Sequence: WP_011883478.1), Mycoplasma mobile (e.g., NCBI Reference Sequence: WP_041362727.1), Neisseria cinerea (e.g., NCBI Reference Sequence: WP_003676410.1), Neisseria meningitidis (e.g., GeneBank: ODP42304.1), Nitratifractor salsuginis (e.g., NCBI Reference Sequence: WP_083799866.1), Parvibaculum lavamentivorans (e.g., NCBI Reference Sequence: WP_011995013.1), Pasteurella multocida (e.g., GeneBank: KUM14477.1), Sphaerochaeta globusa (e.g., NCBI Reference Sequence: WP_013607849.1), Streptococcus pasteurianus (e.g., NCBI Reference Sequence: WP_061100419.1), Streptococcus thermophilus (e.g., GeneBank: ANJ62426.1), Sutterella wadsworthensis (e.g., NCBI Reference Sequence: WP_005430658.1), and Treponema denticola (e.g., NCBI Reference Sequence: WP_002684945.1).

As used herein, the term “functional variants” includes polypeptides which are about 70% identical, at least about 80% identical, at least about 90% identical, at least about 95% identical, at least about 98% identical, at least about 99% identical, at least about 99.5% identical, or at least about 99.9% identical to a protein's native amino acid sequence (i.e., wild-type amino acid sequence) and which retain functionality.

The term “functional variants” also includes polypeptides which are shorter or longer than a protein's native amino acid sequence by about 5 amino acids, by about 10 amino acids, by about 15 amino acids, by about 20 amino acids, by about 30 amino acids, by about 40 amino acids, by about 50 amino acids, by about 75 amino acids, by about 100 amino acids or more and which retain functionality.

The term “functional variants” also includes fusion proteins which retain functionality (e.g., fusion proteins that contain the binding domain of a CRISPR/Cas protein). The term “fusion protein” refers to the combination of two or more polypeptides/peptides in a single polypeptide chain. Fusion proteins typically are produced genetically through the in-frame fusing of the nucleotide sequences encoding for each of the said polypeptides/peptides. Expression of the fused coding sequence results in the generation of a single protein without any translational terminator between each of the fused polypeptides/peptides. Alternatively, fusion proteins also can be produced by chemical synthesis.

The term “retain functionality” refers to a CRISPR/Cas protein variant's ability to bind RNA and cleave polynucleic acids at least about 5%, 10%, 20%, 25%, 30%, 40%, 50%, 60%, 70%, 75%, 80%, 90%, 100%, or more than 100% as efficiently as the respective nonvariant (i.e., wild-type) CRISPR/Cas protein. Methods of measuring and comparing the efficiency of RNA binding and polynucleic acid cleavage are known to those skilled in the art.

As used herein, the term “guide RNA” refers to a polynucleic acid molecule that has a sequence that complements a guide RNA target site, which mediates binding of the CRISPR/Cas complex to the guide RNA target site, providing the specificity of the CRISPR/Cas complex. Typically, guide RNAs that exist as single RNA species comprise two domains: (1) a “guide” domain that shares homology to a target nucleic acid (e.g., directs binding of a CRISPR/Cas complex to a target site); and (2) a “direct repeat” domain that binds a CRISPR/Cas protein. In this way, the sequence and length of a small guide RNA may vary depending on the specific guide RNA target site and/or the specific CRISPR/Cas protein (Zetsche et al. Cell 163, 759-71 (2015)). In some embodiments, the guide RNA may be constructed of DNA, a mixture of DNA and RNA, and/or use modified non-canonical bases. The term “guide RNA target site” refers to sequence that a guide RNA is designed to complement.

As used herein, the term “double stranded oligonucleotide” refers to a double stranded polynucleic acid molecule that is capable of being ligated to another polynucleic acid molecule. The length of the double stranded oligonucleotide can vary. In some embodiments, the double stranded oligonucleotide is between about 5 and 10 nucleotides in length. In other embodiments, the double stranded oligonucleotide is between about 10 and 100 nucleotides in length. In other embodiments, the double stranded oligonucleotide is greater than 100 nucleotides in length.

The abundance of modified nucleotides that a double-stranded oligonucleotide comprises may vary. For example, in some embodiments, less than 25% of the nucleotides in a double-stranded oligonucleotide are modified nucleotides. In other embodiments, at least 25%, at least 30%, at least 40%, at least 50%, at least 60%, at least 70%, at least 80%, at least 90%, or 100% of the nucleotides in the double-stranded oligonucleotide are modified nucleotides.

Enrichment of a polynucleotide region of interest can be facilitated by using Cpf1-mediated double-strand cleavage of target regions to create 5′ overhangs, followed by filling the overhang ends of DNA using modified, nuclease-resistant nucleotides and an appropriate polymerase, or by ligation of an oligonucleotide that contains modified, nuclease-resistant nucleotides.

A polynucleic acid molecule containing one or more target polynucleotide regions of interest is contacted with Cpf1 and guide RNAs (gRNAs) that contain sequences specific for the sequences flanking the target regions. The Cpf1 then makes double-strand cuts in the polynucleic acid molecule at the specific sequences, resulting in five-nucleotide 5′ overhangs at the ends of the polynucleic acid molecule flanking the target regions. Portions of the polynucleic acid molecule that do not contain the target regions will not be cut, or will have only one end cut. The polynucleic acid molecule containing one or more target regions is then protected from exonuclease digestion by filling in the 3′ strand of the overhang with modified, nuclease-resistant nucleotides. This fill-in reaction can be performed by standard polymerase-mediated synthesis, such as by performing an extension reaction with the Klenow fragment of DNA Polymerase I. The nucleotides used to fill in the overhang typically are a mixture of at least one type of modified, nuclease-resistant nucleotide and at least one type of unmodified or nuclease-sensitive nucleotide, such as a combination of naturally-occurring unmodified deoxynucleotide triphosphates (dATP, dTTP, dCTP and dGTP) and modified thiol-containing deoxynucleotide triphosphates (aS-dATP, aS-dTTP, aS-dCTP, aS-dGTP). However, while not preferable, it also is possible to use zero unmodified or nuclease-sensitive nucleotides, depending on the base content of the overhang that is to be left unprotected to exonuclease digestion. Moreover, if no bases are filled in on the overhang, the overhang will be digestible by exonucleases.

Once the overhang is filled in with at least one type of modified, nuclease-resistant nucleotide, the polynucleic acid molecules are then exposed to an exonuclease that is capable of digesting polynucleic acid molecules with unmodified or nuclease-sensitive nucleotides in a 3′ to 5′ manner and substantially less capable of digesting polynucleotide strands with incorporation of modified, nuclease-resistant nucleotides at the 3′ end. Thus only polynucleic acid molecules that have both ends filled in with modified, nuclease-resistant nucleotides will not be digested. These protected molecules will contain the target regions of interest.

The Cpf1 cut sites can be selected such that only target regions are flanked by Cpf1 cuts, and such that the overhangs to be filled in have one or more selected base types. For example, a targeted region could be selected with two distinct Cpf1/gRNA complexes that bind to and cut at sequences flanking the targeted region to produce 5′ overhangs that contain only a single type of base, such as only C bases. The complementary overhangs present in the termini of the fragments separated from the target region would therefore only have a single type of base complementary to the selected bases in the 5′ overhang, such as only G bases in the case of only C bases in the 5′ overhang. The nucleotide mix used to fill in the 5′ overhangs is selected so that only the 5′ overhang is filled in with nuclease-resistant nucleotides. Using the example of only C bases in the 5′ overhang, a nucleotide mixture of nuclease-resistant phosphorothioated dGTP and unmodified, nuclease-sensitive dCTP, dTTP and dATP would result in filling in the flanking 5-base 5′ overhangs with up to five consecutive phosphorothioated dGTPs added to each 3′ end, which provides protection from subsequent digestion with an exonuclease. In contrast, the complementary overhangs (of the off-target regions) are filled in with unmodified, nuclease-sensitive dCTPs, which provides no protection from subsequent digestion with an exonuclease.

Similarly, if the 5′ overhangs flanking the target regions are selected to contain only C and A bases, then protection from exonuclease digestion can be conferred by filling in the overhangs using mixtures of nucleotides that contain (for DNA) modified, nuclease-resistant dGTP and/or modified, nuclease-resistant dTTP, and nuclease-sensitive (such as unmodified) other nucleotides. Modified dNTPs that are digestible by a selected exonuclease can be used instead of unmodified dNTPs, such as dideoxy nucleotide triphosphates, haptenated nucleotides, etc. The nuclease-resistant and nuclease-sensitive dNTPs are selected to give maximum protection to the region of interest while minimizing off-target protection.

As an alternative to filling in overhangs using a polymerase reaction, synthetic double strand linkers containing nuclease-resistant nucleotides can be ligated to the overhangs flanking the selected target regions of interest in the polynucleic acid molecules, such as those cut by Cpf1. The linkers preferably are double stranded with one end having a 5′ overhang sequence complementary to the 5′ overhang sequence generated by theCpf 1 cut. Alternatively one or more single-stranded oligonucleotides containing nuclease-resistant nucleotides can be used, in which the single-stranded oligonucleotides are complementary to the 5′ overhangs.

The linkers contain nuclease-resistant nucleotides. Once ligated onto the end of the Cpf1-generated target molecule, the nuclease-resistant nucleotides make the target sequence resistant to exonuclease digestion. In addition to the sequence needed for hybridizing to the overhang, the linkers can include other sequences (e.g. PCR primer sequences) and/or haptens into the linkers selected by the user for downstream fragment analysis or manipulation.

Alternatively to using a Cpf1 or scCas9 endonuclease to create one or more overhangs, the same effect can be achieved by using nicking endonucleases to make two nicks, one on each strand, in close proximity. Nicking endonucleases can include engineered Cas9 nickases (also referred to as nCas9 or Cas9n), such as Cas9 having an inactivating mutation in either the HNH domain or RuvC domain active sites (e.g., D10A or H840A); naturally occurring or variant endonucleases such as Nt.CviPII; Nb.BssSI, Nt.BspQI, Nt.CviPII, Nt.BstNBI, Nb.BsrDI, Nb.Btsl, Nt.AlwI, Nb.BbvCI, Nt.BbvCI, Nb.Bsml, Nt.BsmAI (all available from New England Biolabs); HNHE, gp74 of HK97, gp37 of Φ SLT, Φ12 HNHE, I-PfoP3I, I-Ts1I; and homing endonucleases (HEases) such as I-Hmul. See, e.g., Chan et al., Nucleic Acids Res. 2011 January; 39(1): 1-18; Xu, Biomol Concepts. 2015 August; 6(4):253-67; Mali et al. Nat Biotechnol. 2013 September; 31(9):833-8; Ran et al., Cell. 2013 Sep. 12; 154(6):1380-9.

Engineered Cas9 nickases can be used by targeting two CRISPR/Cas complexes with two independent guide RNAs. Each guide RNA is designed to recognize a sequence in close proximity to the sequence recognized by the other guide RNA, with one guide RNA targeting the sense strand and the other guide RNA targeting the antisense strand of the desired location in the polynucleic acid molecule.

Other nickases can be used similarly by selecting appropriate sets of nickases to create nicks on both strands in close proximity, thereby creating overhangs.

### EXAMPLES

### Example 1. End Protection-Mediated Polynucleotide Enrichment

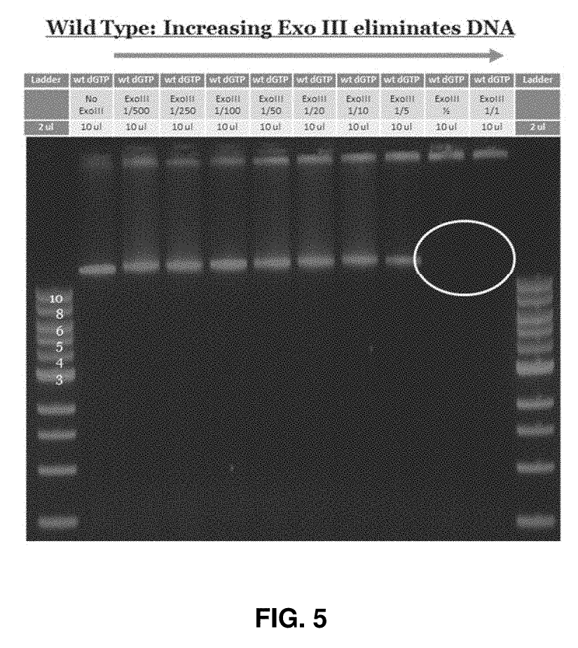

Enrichment of a polynucleotide region of interest can be facilitated by filling 3′ overhang ends of DNA using modified nucleotides. The ends of Lambda DNA have 12-base 5′ overhangs; thus, the 3′ strand can be filled in with modified bases. To demonstrate the utility of this approach, an extension reaction with Klenow enzyme on stock Lambda DNA template was performed using dATP, dTTP, dCTP and either dGTP or S-dGaS-TP modified bases. The extended samples were then exposed to Exonuclease III and resolved on a gel (FIG. 5). Incorporation of modified nucleotides protects the extended Lambda DNA from nuclease-mediated digestion.

### Example 2. CRISPR-Directed End-Protection Mediated Polynucleotide Enrichment

Enrichment of a polynucleotide region of interest can be facilitated by using Cpf1-mediated double-strand cleavage of target regions followed by filling 5′ overhang ends of DNA using modified nucleotides and an appropriate polymerase.

Cpf1 is an RNA-guided endonuclease of the class II CRISPR/Cas system, capable of making double-strand breaks in a site-specific manner. Direction to specific sites in the target region is guided by synthetic RNAs (gRNAs) that contain sequences specific for the target regions as well as sequences needed for binding to Cpf1. The Cpf1 then cleaves the target double-strand DNA resulting in five-nucleotide 5′ overhangs at the ends of the DNA. The 3′ strand of the overhang is then filled in with modified bases using an extension reaction with Klenow enzyme and a combination of naturally-occurring deoxynucleotide triphosphates (dATP, dTTP, dCTP and dGTP) and modified thiol-containing deoxynucleotide triphosphates (aS-dATP, aS-dTTP, aS-dCTP, aS-dGTP). These are also referred to as dNTPs herein.

The filled-in DNA molecules are then exposed to Exonuclease III, which is capable of digesting DNA with unmodified nucleotides in a 3′ to 5′ manner and substantially less capable of digesting polynucleotide strands with incorporation of modified nucleotides at the 3′ end.

By carefully selecting the Cpf1 cut site, the base type content of the overhangs to be filled in can be pre-determined. For example, a targeted region could be selected with two distinct Cpf1/gRNA complexes that bind to and cut at sequences flanking the targeted region to produce 5′ overhangs that contain only C bases. The complementary overhangs would be the termini of the fragments separated from the target region and would have only G bases. In this example, the dNTP mix used to fill in the 5′ overhangs would include the phosphorothioated dGTP and unmodified dCTP, dTTP and dATP. The flanking 5-base overhangs would then have up to five consecutive phosphorothioated dNTPs added to each 3′ end, which provides protection from subsequent digestion with Exonuclease III. The complementary overhangs (of the off-target regions) created by Cpf1 digestion are filled in with unmodified dCTP, providing no protection from subsequent digestion with Exonuclease III.

Similarly, if the 5′ overhangs flanking the target regions are selectedto contain only C and A bases, then the following mixes would provide protection via the G and/or T dNTPs incorporated into the flanking 5′ overhangs, while the complementary overhangs would not be protected:

| Modified dNTPs | Unmodified dNTPs |
|---|---|
| S-dGTP | dCTP, dATP, dTTP |
| αS-dGTP, αS-dTTP | dCTP, dATP |
| S-dTTP | dCTP, dATP, dGTP |

Alternatively, modified dNTPs that are digestible by a selected exonuclease, for example dideoxy nucleotide triphosphates or haptenated nucleotides, can be used instead of unmodified dNTPs in the scheme described above. Also, other modified dNTPs that are resistant to a selected exonuclease can be used instead of phosphorothioate dNTPs in the scheme described above.

The modified and unmodified dNTPs may be selected to give maximum protection to the region of interest while minimizing off-target protection. Selecting nucleotides is based on creating five-nucleotide fill in reactions with modified nucleotides resistant to the nuclease selected to degrade unprotected ends (e.g., Exonuclease III), while adjacent regions are filled in with unmodified nucleotides (or modified nucleotides that are not resistant to the selected nuclease).

As another alternative, synthetic double strand linkers can be ligated to the ends of the DNA molecules cut by Cpf1. The linkers are double stranded with one end having a 5′ overhang sequence complementary to the 5′ overhang sequence generated by the Cpf1 cut. The linkers are synthesized such that the ligated linker includes phosphorothioated bases (or other modified nucleotides resistant to the nuclease selected to degrade unprotected ends), such as at the 3′ terminal end. Once ligated onto the end of the Cpf1-generated target molecule the phosphorothioated bases make the target sequence resistant to exonuclease digestion. This approach also allows the end user to incorporate other sequences (e.g. PCR primer sequences) and/or haptens into the linkers for downstream fragment analysis or manipulation.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### OTHER EMBODIMENTS

All of the features disclosed in this specification may be combined in any combination.

Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present invention are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present invention.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A method for isolating a target nucleic acid, the method comprising:
hybridizing at least one primer to a polynucleotide sequence flanking a target nucleic acid in a sample;
extending the primer using a polymerase and modified nucleotides that are resistant to degradation to create a modified polynucleotide;
exposing the sample to a nuclease; and
isolating the modified polynucleotide.

2. The method of claim 1, further comprising exposing the sample to a selective nuclease that generates at least one double-stranded break comprising an overhang prior to hybridization; wherein the polymerase fills in at least a portion of the overhang with modified nucleotides to create the modified polynucleotide during extension.

3. The method of claim 2, wherein the selective nuclease is selected from the group consisting of a methylation specific nuclease, a methylcytosine-specific endonuclease, a mismatch excision nuclease, a uracil excision nuclease, an abasic site nuclease, a restriction enzyme, and a sequence dependent nuclease.

4. The method of claim 3, wherein the modified nucleotides comprise modified nucleotide triphosphates, alpha-phosphorothioate nucleotide triphosphates, morpholino triphosphates, peptide nucleic acids, peptide nucleic acid analogs, or sugar modified nucleotide triphosphates.

5. The method of claim 4, wherein the modified nucleotides are selected from the group consisting of 2′-Deoxycytidine-5′-O-(1-Thiotriphosphate), 2′-O-methyl modified nucleotide triphosphate, 2′-fluoro modified nucleotide, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, 2′-O-Methylinosine-5′-Triphosphate, 2′-O-Methyl-2-aminoadenosine-5′-Triphosphate, 2′-O-Methylpseudouridine-5′-Triphosphate, 2′-O-Methyl-5-methyluridine-5′-Triphosphate, 2′-O-Methyl-N6-Methyladenosine-5′-Triphosphate, 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-Fluoro-thymidine-5′-Triphosphate, 2′-Deoxyadenosine-5′-O-(1-Thiotriphosphate), 2′-Deoxycytidine-5′-O-(1-5 Thiotriphosphate), 2′-Deoxyguanosine-5′-O-(1-Thiotriphosphate), 2′-Deoxythymidine-5′-O-(1-Thiotriphosphate), Adenosine-5′-O-(1-Thiotriphosphate), Cytidine-5′-O-(1-Thiotriphosphate), Guanosine-5′-O-(1-Thiotriphosphate), Uridine-5′-O-(1-Thiotriphosphate), 2′,3′-Dideoxyadenosine-5′-O-(1-Thiotriphosphate), 2′,3′-Dideoxycytidine-5′-O-(1-Thiotriphosphate), 2′,3′-Dideoxyguanosine-5′-O-(1-Thiotriphosphate), 3′-Deoxythymidine-5′-10 O-(1-Thiotriphosphate), 3′-Azido-2′,3′-dideoxythymidine-5′-O-(1-Thiotriphosphate), 2′,3′-Dideoxyuridine-5′-O-(1-Thiotriphosphate), 2′-Deoxyadenosine-5′-O-(1-Boranotriphosphate), 2′-Deoxycytidine-5′-O-(1-Boranotriphosphate), 2′-Deoxyguanosine-5′-O-(1-Boranotriphosphate), and 2′-Deoxythymidine-5′-O-(1-Boranotriphosphate).

6. The method of claim 5, wherein the modified polynucleotide comprises at least one phosphorothioate linkage, N3′ phosphoramidate linkage, boranophosphate internucleotide linkage, or phosphonoacetate linkage.

7. The method of claim 6, wherein natural nucleotides are used in combination with modified nucleotides.

8. The method of claim 7, wherein the nuclease comprises an exonuclease.

9. The method of claim 8, wherein the sample is a blood sample, serum sample, plasma sample, urine sample, saliva sample, semen sample, feces sample, phlegm sample, or liquid biopsy.

10. A method for isolating a target nucleic acid, the method comprising:
cleaving, in a sequence-specific manner, a polynucleotide sequence flanking a target nucleic acid in a sample to generate at least one double-stranded break flanking the target nucleic acid;
linking modified nucleotides that are resistant to degradation to an overhang of the double-stranded break to create a modified polynucleotide;
exposing the sample to a nuclease; and
isolating the modified polynucleotide.

11. The method of claim 10, wherein linking the modified nucleotides comprises hybridizing at least one primer to the overhang, and extending the primer using a polymerase and the modified nucleotides to create the modified polynucleotide.

12. The method of claim 10, wherein linking the modified nucleotides comprises ligating an oligonucleotide comprising the modified nucleotides to the overhang to create the modified polynucleotide.

13. The method of claim 10, wherein the cleaving is performed by a Cas endonuclease complexed with a guide RNA that targets the Cas endonuclease to a region flanking the target nucleic acid.

14. The method of claim 13, wherein the modified nucleotides comprise modified nucleotide triphosphates, alpha-phosphorothioate nucleotide triphosphates, morpholino triphosphates, peptide nucleic acids, peptide nucleic acid analogs, or sugar modified nucleotide triphosphates.

15. The method of claim 14, wherein the modified nucleotides are selected from the group consisting of 2′-Deoxycytidine-5′-O-(1-Thiotriphosphate), 2′-O-methyl modified nucleotide triphosphate, 2′-fluoro modified nucleotide, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, 2′-O-Methylinosine-5′-Triphosphate, 2′-O-Methyl-2-aminoadenosine-5′-Triphosphate, 2′-O-Methylpseudouridine-5′-Triphosphate, 2′-O-Methyl-5-methyluridine-5′-Triphosphate, 2′-O-Methyl-N6-Methyladenosine-5′-Triphosphate, 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-Fluoro-thymidine-5′-Triphosphate, 2′-Deoxyadenosine-5′-O-(1-Thiotriphosphate), 2′-Deoxycytidine-5′-O-(1-5 Thiotriphosphate), 2′-Deoxyguanosine-5′-O-(1-Thiotriphosphate), 2′-Deoxythymidine-5′-O-(1-Thiotriphosphate), Adenosine-5′-O-(1-Thiotriphosphate), Cytidine-5′-O-(1-Thiotriphosphate), Guanosine-5′-O-(1-Thiotriphosphate), Uridine-5′-O-(1-Thiotriphosphate), 2′,3′-Dideoxyadenosine-5′-O-(1-Thiotriphosphate), 2′,3′-Dideoxycytidine-5′-O-(1-Thiotriphosphate), 2′,3′-Dideoxyguanosine-5′-O-(1-Thiotriphosphate), 3′-Deoxythymidine-5′-10 O-(1-Thiotriphosphate), 3′-Azido-2′,3′-dideoxythymidine-5′-O-(1-Thiotriphosphate), 2′,3′-Dideoxyuridine-5′-O-(1-Thiotriphosphate), 2′-Deoxyadenosine-5′-O-(1-Boranotriphosphate), 2′-Deoxycytidine-5′-O-(1-Boranotriphosphate), 2′-Deoxyguanosine-5′-O-(1-Boranotriphosphate), and 2′-Deoxythymidine-5′-O-(1-Boranotriphosphate).

16. The method of claim 15, wherein the modified polynucleotide comprises at least one phosphorothioate linkage, N3′ phosphoramidate linkage, boranophosphate internucleotide linkage, or phosphonoacetate linkage.

17. The method of claim 16, wherein natural nucleotides are used in combination with modified nucleotides.

18. The method of claim 17, wherein the nuclease comprises an exonuclease.

19. The method of claim 18, wherein the sample is a blood sample, serum sample, plasma sample, urine sample, saliva sample, semen sample, feces sample, phlegm sample, or liquid biopsy.

20. A method for isolating a target nucleic acid, the method comprising:
binding an epigenetic-binding moiety to a polynucleotide sequence within or flanking target nucleic acids in a sample to sterically inhibit nuclease degradation of the target nucleic acids;
exposing the sample to a nuclease; and
isolating the target nucleic acids.

21. The method of claim 20, wherein the epigenetic-binding moiety comprises a protein or an antibody.

22. The method of claim 21, wherein the epigenetic-binding moiety comprises methyl-cytosine binding proteins or methyl-cytosine binding antibodies.

23. The method of claim 22, wherein the nuclease comprises an exonuclease.

24. The method of claim 23, wherein the sample is a blood sample, serum sample, plasma sample, urine sample, saliva sample, semen sample, feces sample, phlegm sample, or liquid biopsy.

25. A method for isolating a target nucleic acid, the method comprising:
dephosphorylating a polynucleotide having at least one double-stranded break flanking a target nucleic acid in a sample to protect the target nucleic acid from nuclease degradation;
exposing the sample to a nuclease; and
isolating the target nucleic acid.

26. The method of claim 25, further comprising cleaving, in a sequence-specific manner, a polynucleotide sequence flanking the target nucleic acid in the sample to generate the at least one double-stranded break prior to dephosphorylation.

27. The method of claim 26, wherein the cleaving is performed by a Cas endonuclease complexed with a guide RNA that targets the Cas endonuclease to a region flanking the target nucleic acid.

28. The method of claim 27, wherein the nuclease comprises an exonuclease.

29. The method of claim 28, wherein the sample is a blood sample, serum sample, plasma sample, urine sample, saliva sample, semen sample, feces sample, phlegm sample, or liquid biopsy.
